(19) United States
(12) Patent Application Publication
(10) Pub. No.: US 2018/0235903 A1
(43) Pub. Date: Aug. 23, 2018

(54) Fentanyl Transdermal Delivery System

(71) Applicant: Amneal Pharmaceuticals LLC, Bridgewater, NJ (US)

(72) Inventors: Jay Audett, Somerset, NJ (US); Chee Youb Won, Belle Mead, NJ (US)

(21) Appl. No.: 15/557,637

(22) PCT Filed: Mar. 11, 2016
(86) PCT No.: PCT/US2016/021961

§ 371 Date: Sep. 12, 2017

Related U.S. Application Data
(60) Provisional application No. 62/132,666, filed on Mar. 13, 2015.

(51) Int. Cl. A61K 9/70; A61K 9/00; A61K 31/4468; A61F 13/00; A61F 13/02

(52) U.S. Cl. CPC A61K 9/7069; A61K 9/0014; A61K 31/4468; A61F 13/0253; A61K 9/7053; A61F 13/00063; A61K 9/7061

(57) ABSTRACT

Described is a transdermal device comprising a backing layer; an adhesive matrix comprising fentanyl as an active agent suspended on a plurality of pressure sensitive adhesives along with a cross-linked moisture absorbent comprising cross-linked polyvinylpyrrolidone; and a release layer. Also described is a method of relieving pain, which comprising applying said transdermal device to the skin of a patient in need thereof.

### TECHNICAL FIELD

The present invention relates generally to the field of transdermal drug delivery. More particularly, the invention relates to fentanyl-based, silicone and polyacrylate pressure sensitive adhesive formulations, and their use in making devices for improved transdermal delivery of fentanyl.

### BACKGROUND

Transdermal delivery of various drugs is well known in the art of drug delivery. Pressure sensitive adhesive matrix patches are also known and typically include an inert, impervious backing layer, a pressure sensitive adhesive layer containing the drug and optional selected excipients, and a release liner that is peeled off and discarded before applying the patch to the skin. Transdermal drug delivery patches dispense a drug at a controlled rate by presenting the drug for absorption in an efficient manner, for example, with a minimal residual drug or degradation of the drug, and prevent complications from failure of a patient to comply with a therapeutic regimen. The pressure sensitive adhesive layers may exhibit “cold flow,” the flow of the adhesives from underneath the backing layer/out of the edge of the patch during storage or wear by the patient. FIG. 1 illustrates a conventional transdermal patch 100 including a release liner 102, drug particles 110 in an adhesive matrix 104, and a backing layer 106. As illustrated in FIG. 1, the transdermal patch 100 has significant cold flow 108, flowing/seeping out onto the release liner 102. As illustrated, because the cold flow seeps outside of the adhesive matrix 104 and the backing layer 106, the cold flow 108 remains on the release liner when the release liner is removed. Cold flow could lead to therapeutic and safety risk, that is, improper delivery of fentanyl, which is a very potent opioid due to a change in surface area created by the adhesive cold flow. The occurrence of cold flow during storage can lead to a shorter shelf-life, and/or to a residue of adhesive being left behind on the patient's skin following patch removal and/or to decreased patient compliance. It is desirable that the patches themselves are comfortable on the skin, which may be measured indirectly by adhesive rheology, or through tack, shear strength, and peel adhesion measurements. Hence, it is challenging to formulate a transdermal patch that reduces its cost and size while providing excellent comfort with minimal cold flow.

Fentanyl, which is an opioid analgesic with a rapid onset and short duration of action, has historically been used as a general anesthetic and to treat pain. Fentanyl is a very potent analgesic, and, because of its potency, has been used with transdermal delivery systems, e.g. transdermal patches, for pain relief, particularly chronic pain relief. The different transdermal delivery systems used by various different companies affect individual rates of adsorption of the fentanyl. Typically, a fentanyl patch is available either as a reservoir membrane patch or a matrix patch. The reservoir membrane patch typically consists of a backing layer, an absorbent pad or a drug reservoir surrounded by an adhesive and a release liner. The matrix patch contains of a protective release liner, a drug in adhesive matrix layer, and a backing layer.

Denpax®, supplied by Alphapharm Pty Limited, is a monolithic, drug-in-adhesive transdermal drug delivery system, where the active agent, fentanyl, is dispersed within the adhesive matrix of the patch. The patch includes a polyolefin backing layer with white imprinting ink on the uncoated side and a fentanyl (4.0% w/w) containing silicone adhesive layer (89.5% w/w) with Dimethicone 360 (6.5% w/w). Before use, a protective fluorocarbon-coated release liner that is attached to and covering the adhesive layer is removed and discarded. Denpax® transdermal drug delivery systems are packaged with additional pieces of protective fluorocarbon coated polyester release liner films above and below the system within each pouch; these release liners are discarded at the time of use. In the case of Denpax®, variations in skin temperature affect the delivery rate of the active agent due to the changes in the skin permeability.

U.S. Pat. No. 8,449,907 B2 to Mylan Pharmaceuticals describes a process for manufacturing fentanyl transdermal patches, wherein fentanyl particle size is about 10 to 20 microns. The formulation is made by blending fentanyl particles directly with one or more solvated silicone adhesives (87.50% w/w) to form a suspension of fentanyl particles in the solvated silicone adhesive(s), and dimethicone (6.5% w/w) silicone oil blend. The formulation is plasticized by the dimethicone silicone oil. The backing material is elastomeric.

U.S. Patent Application Publication No. 2008/0226698 A1 to Mylan Technologies, Inc., describes a transdermal drug delivery system comprising a backing layer, an adhesive matrix layer comprising a supersaturated concentration of an active agent that is present in an amorphous form within an adhesive matrix, and a release liner. The adhesive matrix includes at least one cohesive enhancer, such as polyvinylpyrrolidone or Crospovidone. The backing layer ranges from 1.0 mil to at least 3.0 mil.

There is an on-going need to provide transdermal drug delivery systems with minimal cold flow properties or residual drug, and provide drug delivery at an acceptable fentanyl skin flux.

### SUMMARY

A first aspect of the invention pertains to a transdermal delivery system. In a first embodiment, a transdermal delivery system comprises: a backing layer; an adhesive matrix comprising fentanyl, an analgetically effective relative of fentanyl, or mixtures thereof, a plurality of pressure sensitive adhesives selected from a silicone adhesive, a polyacrylate adhesive, and combinations thereof, and a cross-linked moisture absorbent comprising cross-linked polyvinylpyrrolidone; and a release liner.

In a second embodiment, the transdermal delivery system the first embodiment is modified, wherein the fentanyl comprises crystals having a particle size (d₅₀) in a range of about 5 to 20 microns as measured by laser diffraction.

In a third embodiment, the transdermal delivery system of the first and second embodiments is modified, wherein the particle size (d₅₀) is in a range of about 10 to about 15 microns

In a fourth embodiment, the transdermal delivery system of the first through third embodiments is modified, wherein the polyacrylate adhesive is present in the adhesive matrix in an amount in a range of about 1.0 to about 4.0% w/w.

In a fifth embodiment, the transdermal delivery system of the first through fourth embodiment is modified, wherein the silicone adhesive is present in the adhesive matrix in an amount in a range of about 80 to about 90% w/w.

In a sixth embodiment, the transdermal delivery system of the first through fifth embodiments is modified, wherein the fentanyl is present in the adhesive matrix in an amount in a range of about 3 to about 5% w/w.

In a seventh embodiment, the transdermal delivery system of the first through sixth embodiments is modified, wherein the cross-linked polyvinylpyrrolidone is present in the adhesive matrix in an amount in a range of about 2 to about 6% w/w.

In an eighth embodiment, the transdermal delivery system of the first through seventh embodiments is modified, wherein the silicone adhesive further comprises a silicone medical fluid.

In a ninth embodiment, the transdermal delivery system of the eighth embodiment is modified, wherein the silicone medical fluid comprises dimethicone.

In a tenth embodiment, the transdermal delivery system of the ninth embodiment is modified, wherein the dimethicone is present in the adhesive matrix in an amount in a range of about 2 to about 7% w/w.

In an eleventh embodiment, the transdermal delivery system of the first through tenth embodiments is modified, wherein the release liner comprises one or more of paper, coated paper, plastic films, polyolefins made of high density polyethylene (HDPE), low density polyethylene (LDPE), polypropylene (PP) plastic resin, fluoropolymer-coated films.

In a twelfth embodiment, the transdermal delivery system of the eleventh embodiment is modified, wherein the release liner comprises LDPE.

In a thirteenth embodiment, the transdermal delivery system of the eleventh embodiment is modified, wherein the release liner comprises a fluoropolymer-coated polyethylene terephthalate (PET) film.

In a fourteenth embodiment, the transdermal delivery system of the first through thirteenth embodiments is modified, wherein the backing layer comprises films of polyethylene, polyethylene terephthalate (PET), polypropylene, polyurethane, ethylene vinyl acetate (EVA) of polyamide, metal foils, or paper, alone or coated with a polymeric material, or mixtures thereof.

In a fifteenth embodiment, the transdermal delivery system of the first through fourteenth embodiments is modified, wherein the backing layer comprises a PET-EVA laminate.

In a sixteenth embodiment, the transdermal delivery system of the first through fifteen embodiments is modified, wherein the backing layer has a thickness of less than about 2.5 mil.

In a seventeenth embodiment, the transdermal delivery system of the first through sixteenth embodiments is modified, wherein the transdermal delivery system has reduced cold flow when compared to transdermal delivery systems that do not contain cross-linked polyvinylpyrrolidone.

In an eighteenth embodiment, the transdermal delivery system of the first through seventeenth embodiments is modified, wherein the transdermal delivery system has skin flux in the range of about 2 to about 6 mcg/cm²/h.

A second aspect of the invention relates to a transdermal delivery system. In a nineteenth embodiment, a transdermal delivery system comprises a backing layer; an adhesive matrix comprising: 3.5 to 4.5% w/w fentanyl, 80 to 90% w/w silicone adhesive, 1 to 4% w/w polyacrylate adhesive, and 2 to 6% w/w cross-linked polyvinylpyrrolidone; and a release liner.

In a twentieth embodiment, the transdermal delivery system of the nineteenth embodiment is modified, wherein the adhesive matrix further comprises 2 to 7% w/w silicone medical fluid.

A third aspect of the present invention relates to a method of relieving pain. In a twenty-first embodiment, a method of relieving pain comprises applying to the skin of a patient in need thereof the transdermal delivery system of claim 1 or 19.

### BRIEF DESCRIPTION OF DRAWINGS

The disclosure may be more completely understood in consideration of the following detailed description of various embodiments of the disclosure in connection with the accompanying drawings, in which:

FIG. 2 is a top view of a conventional transdermal patch;

FIG. 2 provides a cross-section view of an exemplary transdermal matrix patch; and

FIG. 3 provides a top view of an exemplary transdermal matrix patch.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Provided is a transdermal delivery system for pain relief comprising fentanyl as an active agent dispersed in an adhesive matrix. The transdermal delivery system of one or more embodiments contains less fentanyl, provides better adhesion, has reduced adhesive cold flow, and is no larger than the smallest transdermal patch currently on the market. The transdermal delivery system is available as a matrix patch, and comprises fentanyl crystals dispersed in an adhesive matrix of cross-linked, micronized polyvinylpyrrolidone in a polymer blend of acrylic and silicone polymers. The cross-linked, micronized polyvinylpyrrolidone in a polymer blend of acrylic and silicone polymers promotes fast drying during the coating operation and limits the crystal growth of the fentanyl particles following completion of the drying process. This may extend shelf-life of the product and improve the solubility of the drug.

It is noted that the transdermal system of U.S. Pat. No. 8,449,907 has extreme cold flow. In addition, the transdermal system is sandwiched between two release liner sheets in addition to the release liner that protects the adhesive layer. The additional release liner sheets prevent adhesive cold flow contact with the pouch heat seal layer, a low density polyethylene (LDPE) material. Without the additional release liner film, the transdermal system is difficult to remove from the pouch, and the fentanyl diffuses into the LDPE heat seal following pouching, leading to a decline in fentanyl assay, and a reduced shelf life. Additionally, due to the presence of the silicone adhesive, the transdermal patch does not adhere well during hot weather, or under any conditions where perspiration accumulates under the patch, because the perspiration interferes with the adhesion of the patch. Furthermore, it is noted that the backing layer of the transdermal system of U.S. Pat. No. 8,449,907 is about 3 mil thick, which contributes to poor adhesion because of patch edge vulnerability to peel-off.

An improvement in adhesive rheology, an increase in storage modulus at low frequencies, is required to decrease adhesive cold flow during storage and wear.

Another disadvantage of the currently marketed reservoir transdermal delivery systems occurs when the active agent, fentanyl, is in solution in a mixture of ethanol and water. Leakage in this system results in the active agent coming in contact with the skin over a large area and, as a consequence of this contact, the active agent is absorbed in excessive doses which can be potentially fatal.

Yet another disadvantage of the presently marketed Mylan transdermal drug delivery systems is the two release liner sheets. In order to prevent the adhesive cold flow from contacting the pouch heat deal layer, the transdermal patch is typically secured between two release liners on either side. While this structure tackles the immediate cold flow and shelf-life problem by preventing the patch adhesive layer from contacting the pouch heat seal layer, the structure fails to reduce cold flow during wear. Additionally, the presence of two release liners increases the manufacturing cost and process-time.

Another disadvantage of silicone-based adhesives includes limited effectiveness when exposed to certain chemicals, UV rays, or high temperatures (over 150° F./66° C.). In addition, silicone-based adhesives are more susceptible to oxidation and may darken, lose their tack, and become brittle if overexposed. Also, silicone-based adhesives may turn soft and gummy if plasticized, for example, by the dimethicone silicone oil used to plasticize the adhesive.

Current mechanisms of transdermal delivery of fentanyl fall short in balancing drug delivery at an acceptable fentanyl skin flux, while minimizing the amount of residual drug and cold flow, at the same time improving the tack, shear strength, and peel adhesion. Therefore, there is a need to minimize adhesive cold flow and improve tack, peel adhesion, and shear strength, while producing a transdermal monolithic patch which is as thin as possible. The transdermal delivery systems provided herein to serve multiple purposes such as containing the drug and controlling the release of the drug, while maintaining adhesion to the skin.

With respect to the terms used in this disclosure, the following definitions are provided.

As used herein, the term “transdermal patch” refers to a medicated adhesive patch that is placed on the skin to deliver a specific dose of medication through the skin and into the bloodstream. As recognized by one skilled in the art, because the skin is an effective barrier, only drugs whose molecules are small enough to penetrate the skin can be delivered by a transdermal patch.

As used herein, the term “matrix patch” refers to a transdermal patch which has an inert, impervious backing layer, an active agent or drug for delivery, a semisolid adhesive matrix, and a release liner. The drug is incorporated into the adhesive matrix, such that the drug is located within the adhesive. A matrix patch does not contain an absorbent pad, which simplifies the manufacturing process.

As used herein, the term “fentanyl” refers to a potent, synthetic opioid analgesic with rapid onset and short duration of action. In clinical settings, fentanyl exerts is principle pharmacologic effects on the central nervous system. Its primary actions of therapeutic value are often considered to be analgesia and sedation. Fentanyl is indicated for the management of chronic pain in individuals who require opioid analgesia for pain that is typically unmanageable by lesser means. In particular, fentanyl is often used clinically for the relief of postoperative pain and pain related to cancer.

Fentanyl has the chemical structure of Formula (I):

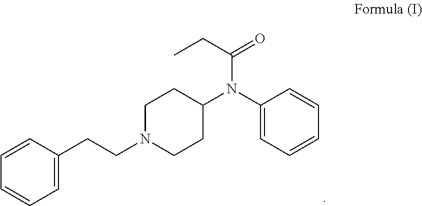 [SMILES: CCC(=O)N(c1ccccc1)C1CCN(CCc2ccccc2)CC1]

Fentanyl is generally administered in the free base form. The quantity of fentanyl contained in the transdermal system is a quantity sufficient to provide a pharmaceutically or physiologically effective dosage rate of the active agent to a patient/host in need thereof.

In one or more embodiments, the fentanyl base can be used in the transdermal delivery system in an amount in the range of about 1% to about 10% w/w (dry weight), including about 3% to about 7% w/w (dry weight), and about 3.5% to about 4.5% w/w (dry weight).

In one or more embodiments, delivery rates of fentanyl will usually be in the range of about 5 to about 250 mcg/hour, including about 10 mcg/hour to about 100 mcg/hour, and including about 12.5 mcg/hour, about 25 mcg/hour, about 37.5 mcg/hour, about 50 mcg/hour, about 62.5 mcg/hour, about 75 mcg/hour, about 87.5 mcg/hour, and about 100 mcg/hour.

In other embodiments, an analgetically effective relative of fentanyl may be administered, including sufentanil, carfentanil, lofentanil, and afentanil.

As used herein, the term “cold flow” refers to the distortion of a solid under sustained pressure, especially with an accompanying inability to return to its original dimensions when the pressure is removed. In other words, cold flow is the tendency of a pressure-sensitive adhesive to exhibit viscous flow over long periods of time.

As used herein, with respect to measuring cold flow, the terms “significant”, “slight”, and “negligible” refer to descriptive standards for cold flow measured microscopically. Wherein, significant is typically 0.3 mm or greater, slight is less than 0.3 mm and greater than 0 and negligible is approximately 0.

As used herein, the term “black border formation” refers to the adhesive cold flow occurring during wear which contacts clothing fibers, lint, or other debris, to form a black border around the transdermal patch.

As used herein, the term “skin flux” refers to the intrinsic flux of a drug/pharmaceutical active ingredient diffusing across a transdermal delivery system to the skin. In other words, skin flux is the amount of a drug/active pharmaceutical ingredient delivered by a transdermal delivery system to permeate the skin. As used herein, the phrase “acceptable skin flux” refers to an amount of drug in the range of about 2 to about 6 mcg/cm²/h, including about 2, about 2.5, about 3, about 3.5, about 4, about 4.5, about 5, about 5.5, and about 6 mcg/cm²/h of drug delivered by the transdermal delivery system to permeate the skin.

As used herein, the term “adhesive matrix” refers to a pressure sensitive adhesive layer, which serves to carry the bioactive substance or drug but also serves to attach the patch to the skin. The adhesive matrix includes an adhesive which is cast onto the material to be used as a release liner or a backing layer.

In one or more embodiments, the adhesive matrix is made in such a manner that components of the adhesive and their solvents are mixed with the drug and/or other substances and then coated on a suitable sheet, intended to function as a disposable liner, and the solvents are removed in a drying process. It is noted that the transdermal patch of one or more embodiments generally has a coating weight that is 95% the weight of currently marketed transdermal patches. Then, a non-releasable backing layer is applied over the adhesive matrix layer. The result is a web-like structure comprised of a pressure-sensitive adhesive matrix layer, containing the drug, sandwiched between a backing layer on one side and a disposable release liner on the other. The web can be cut into suitable sizes and shapes to produce pressure sensitive adhesive transdermal drug delivery patches.

In one or more embodiments, the transdermal patch has a size in the range of 3 to 25 cm², including about 3.13 cm², about 6.25 cm², about 9.38 cm², about 12.50 cm², about 15.63 cm², about 18.75 cm², about 21.88 cm², or about 25 cm².

As used herein, the term “release liner” refers to a film or sheet applied during the manufacture of the transdermal patch used to prevent the adhesive matrix from prematurely adhering to the skin. A release liner is a removable protective sheet that has been rendered “non-stick” to the adhesive matrix. Examples of materials suitable for use as release liners include paper, coated paper, plastic films, polyolefins typically made of high density polyethylene (HDPE), low density polyethylene (LDPE), polypropylene (PP) plastic resin, fluoropolymer-coated films, and silicone-coated films. In one or more embodiments, the release liner comprises LDPE. In other specific embodiments, the release liner comprises a fluoropolymer-coated polyethylene terephthalate (PET) film.

As used herein, the terms “backing” or “backing sheet” or “backing layer” refer to a layer or web applied over the adhesive matrix, which permits transfer of the drug to the wearer, but prevents transfer of the drug to non-wearers. Examples of materials suitable for use as backing layers include films of polyethylene; polyethylene terephthalate (PET); polypropylene; polyurethane; ethylene vinyl acetate (EVA), polyamide; metal foils (e.g. aluminum foil) or paper, alone or coated with a polymeric material, or mixtures thereof. In one or more embodiments, the backing layer comprises a PET-EVA laminate.

In one or more embodiments, the release liner may be the same size as the adhesive matrix layer and/or may be the same size as the backing. In other embodiments, the release liner may be oversized as compared with the adhesive matrix layer. Without intending to be bound by theory, it is thought that use of an oversized release liner may help prevent the adhesive matrix from becoming distorted or relaxing during the shipping and handling processes. In one or more embodiments, the release liner is about 2 to about 3 mil in thickness. In one or more embodiments, the backing layer is a PET-EVA laminate which is approximately 2 mil in thickness (1 mil=0.001 inch).

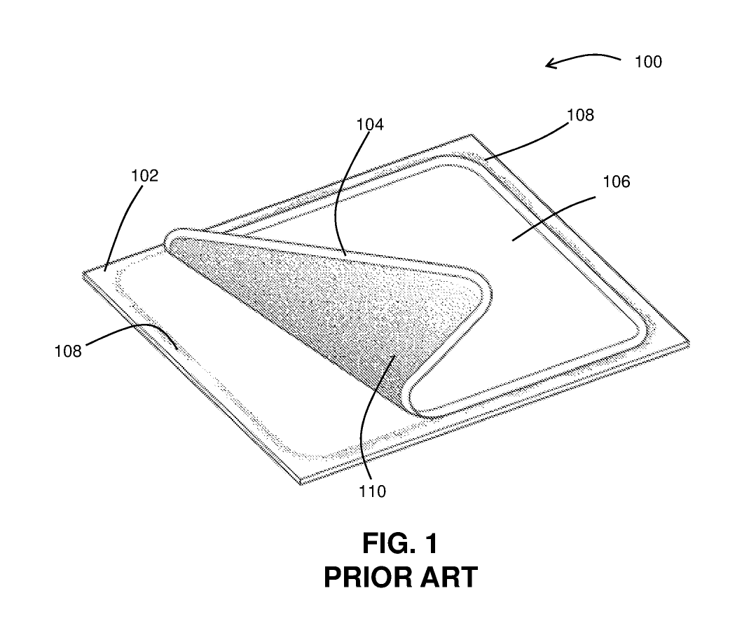

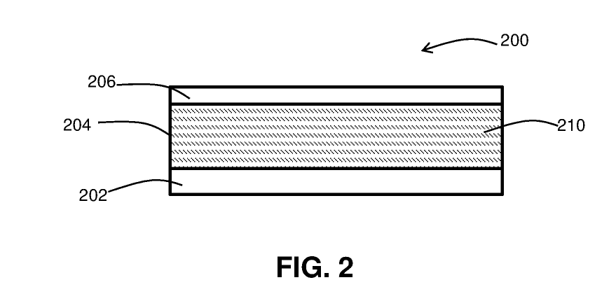

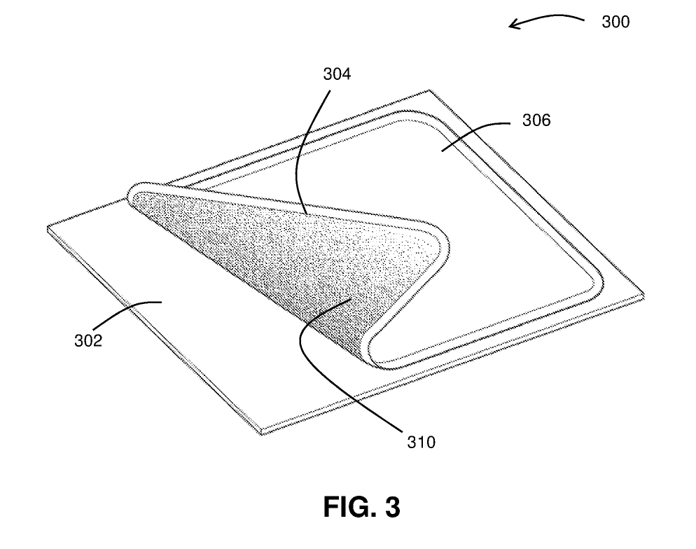

Turning to FIG. 2, a cross-section view of an exemplary transdermal matrix patch is provided. Matrix patch 200 has a backing layer 206 and an adhesive matrix 204 including particles of active agent 210, such as fentanyl, is located on a release liner 202. Turning, to FIG. 3, a topview of an exemplary transdermal matrix patch 300 including a release liner 302, an adhesive matrix 304 including particles of active agent 310, and a backing layer 306. As illustrated in FIG. 3, the transdermal patch 300 does not have significant cold flow.

In one or more embodiments, the transdermal delivery system comprises fentanyl crystals dispersed in an adhesive matrix of cross-linked, micronized polyvinylpyrrolidone (Kollidon® CL-M) and an Acrylic (Duro-Tak® 87-901A)/Silicone (BIO-PSA® 7-4201/360 Silicone medical fluid) polymer blend.

The acrylic polymer and cross-linked, micronized polyvinylpyrrolidone improve the ability of the adhesive to absorb and transmit perspiration, improving adhesion compared to transdermal delivery systems formulated with only silicone adhesive polymers. The cross-linked, micronized polyvinylpyrrolidone is preferred over other cross-linked polyvinylpyrrolidones. The larger particle sizes of the other grades, such as Kollidon® CL-F, yields a less smooth coating because the larger particles appear as lumps in the dried coating. The cross-linked, micronized polyvinylpyrrolidone, Kollidon® CL-M, also plays an important role enabling a uniform dispersion of the acrylic adhesive polymer in the formulated mixture for coating. Without the cross-linked, micronized polyvinylpyrrolidone, Kollidon® CL-M, the acrylic polymer separates from the mixture as large droplets; the addition of the cross-linked, micronized polyvinylpyrrolidone, Kollidon® CL-M, prevents this phase separation by allowing adsorption of the solvated acrylic polymer droplets to the polyvinylpyrrolidone particles.

The inventive adhesive solutions have a high % solids using hydrocarbon solvents such as heptanes for the BIO-PSA® silicone adhesive (approximately 70% solids) and cyclohexane for the acrylic adhesive (Duro-Tak®, approximately 46% solids). The hydrocarbon solvents allow the preparation of mixes with minimal dissolution of the fentanyl base. Excessive dissolution of the fentanyl base can lead to crystallization following removal of the process solvents. This crystallization leads to growth of crystals on the adhesive surface and poor adhesion. The extent of fentanyl dissolution in the mixture can vary between 0.2 to 5.0% by weight to yield dried coatings with minimal fentanyl post-drying crystallization and acceptable adhesion and fentanyl delivery in the finished product.

The transdermal delivery system of one or more embodiments has a fentanyl particle size (d₅₀) in the range of about 8 to about 20 microns, more particularly about 10 to about 15 microns, that is evenly dispersed throughout the adhesive matrix. In one or more embodiments, the fentanyl particle size is determined by laser diffraction. Laser diffraction analysis using a Malvern® Mastersizer 3000 is a technology that utilizes diffraction patterns of a laser beam passed through any object ranging from nanometers to millimeters in size to quickly measure geometrical dimensions of a particle. Furthermore, the transdermal delivery system has a reduced cold flow, which leads to a more effective transdermal patch performance, with improved safety and ease of use. The reduced cold flow of the patch reduces undesirable drug exposure and limits the adhesive residue left behind on the skin following the patch removal.

It has been found that due to the small size of the fentanyl particles, which are suspended in the silicone and polyacrylate adhesive, the level of residual drug following use is reduced. The fine micronized particles of the active agent are more evenly dispersed throughout the patch which promotes reduced cold flow, and acceptable shear strength, tack and peel adhesion. Also, the addition of cross-linked polyvinylpyrrolidone as a moisture absorbent allows maintenance of adhesion onto the skin during periods of heavy perspiration while in no way diminishing the fentanyl skin flux. The polyacrylate which improves the adhesion of the monolithic transdermal patch and the physical properties that go along with it, does not compromise the fentanyl skin flux either.

In one or more embodiments, the fentanyl containing formulations are used to manufacture improved devices for delivering fentanyl transdermally, particularly transdermal patches. The patches are matrix patches. The matrix patch is manufactured by casting the formulation onto a support material such as a backing layer or release liner to form a fentanyl-containing adhesive layer.

In one or more embodiments, the adhesive matrix comprises a polyacrylate and silicone pressure sensitive adhesive formulation including a blend of fentanyl/dispersed suspended in a solvated silicone and polyacrylate pressure sensitive adhesives. The selected solvent is one that can substantially or fully solvate or dissolve the adhesive while keeping the fentanyl suspended/dispersed in the solvated adhesive.

The use of pressure sensitive adhesives (PSAs) such as polyacrylate adhesives and silicone adhesives, which are used to hold the patch to the skin, have desirable properties such as resistance to oxidation, permeability to water vapor and oxygen, good tack behavior and better shear strength; all of which are provided at a moderate cost.

Suitable silicone adhesives include pressure sensitive adhesives made from silicone polymer and resin. Examples of useful silicone adhesives include the BioPSA® series (7-4400, 7-4500, and 7-4600 series) and the amine compatible (end-capped) BioPSA® series (7-4100, 7-4200, and 7-4300 series) manufactured by Dow Corning. In one or more embodiments, BioPSA® 7-4201 is used as the silicone adhesive. The silicone adhesive can be used in an amount in the range of about 80 to about 90 wt. %.

In one or more embodiments, the transdermal patch comprises a cross-linked polyvinylpyrrolidone, which is added as a moisture absorber that allows maintenance of adhesion to the skin, especially during periods of heavy perspiration. Furthermore, the use of cross-linked polyvinylpyrrolidone and an acrylic polymer does not diminish the fentanyl skin flux.

In another embodiment, the cross-linked polyvinylpyrrolidone present in the transdermal patch acts as a cohesive strengthening agent and aids in the absorption of moisture. The use of the PVP does not diminish the delivery of fentanyl from the system.

In yet another embodiment, the cross-linked polyvinylpyrrolidone and an acrylic polymer enable quicker drying with minimal fentanyl crystal growth following completion of solvent removal. The use of acrylic adhesive increases fentanyl solubility in the formulation. The cross-linked polyvinylpyrrolidone facilitates achieving acceptable acrylic polymer uniformity in the mix.

In one or more embodiments, silicone fluids include high molecular weight polydimethylsiloxane, Dimethicone NF (Dow 360 Silicone Medical Fluid, 100 cSt and other viscosities). In specific embodiments, Dimethicone NF is used and is present in an amount of about 0% w/w to about 25% w/w and, more specifically, in a range of about 5% w/w to about 8.5% w/w.

The coat weight of the adhesive matrix layer is that coat weight which provides at least sufficient adhesion of the transdermal delivery system to the skin of the patient/host. The coat weight also may vary depending upon such factors as the amount of drugs to be delivered from the adhesive matrix layer and the desired wear period. The coat weight of the adhesive matrix will usually range from about 9 to 11 mg/cm².

Without intending to be bound by theory, it is believed that the fentanyl patch of one of more embodiments has better solubility than current transdermal fentanyl patches on the market, including those fentanyl transdermal patches product by Mylan Pharmaceuticals. It is thought that the improved solubility is due to the presence of dispersed polyacrylate adhesive on the cross-linked polyvinylpyrrolidone particles present in the adhesive matrix.

In one of the embodiments, the fentanyl transdermal patch comprises a suspension of fentanyl and Kollidon® CL-M in a silicone adhesive, polyacrylate adhesive, and Dimethicone 360 matrix. The use of the cross-linked PVP does not in any way diminish the fentanyl skin flux.

In one or more embodiments the polyacrylate adhesive used, such as DURO-TAK® 87-901, is designed to enable dispersed drug patch formulations. Supplied in non-polar cyclohexane solvent, it is targeted at specific drugs that have low solubility in non-polar solvents, and simplifies preparation of dispersed drug-in-adhesive patches by reducing active pharmaceutical ingredient (API) solubility and diminishing pot-drying crystallization.

In one or more embodiments, the transdermal delivery system has the formulation of components appearing in Table 1. All values are % (w/w) on a dry basis.

TABLE 1
Composition % (w/w)

| Component | Range | Preferred |
|---|---|---|
| Fentanyl Base | 3.5-4.5% | 4.0% |
| Silicone Adhesive | 80-90% | 85.75% |
| BIO-PSA ® 7-4201 | | |
| Silicone Medical Fluid | 2-7% | 4.5% |
| 360 Medical Fluid | | |
| Micronized Cross-Linked | 2-6% | 4.0% |
| Polyvinylpyrrolidone | | |
| Kollidon ® CL-M | | |
| Acrylic Adhesive | 1.0-4.0% | 1.75% |
| Duro-Tak ® 87-901 | | |

The invention is now described with reference to the following examples. Before describing several exemplary embodiments of the invention, it is to be understood that the invention is not limited to the details of construction or process steps set forth in the following description. The invention is capable of other embodiments and of being practiced or being carried out in various ways.

### EXAMPLES

### Example 1: Preparation of Transdermal Patch

The fentanyl powder was weighed inside an isolator and wetted/mixed with approximately one third of the total silicon BIO-PSA® adhesive. The adhesive-solvated fentanyl was transferred into a 2 Gallon Ross Mixer. The remaining fentanyl was subsequently wetted with another third of the total BIO-PSA®, then transferred into the 2 Gallon Ross Mixer. The final third of the BIO-PSA® was used to wet any remaining fentanyl powder which was also added to the 2 Gallon Ross Mixer. Any remaining BIO-PSA® and fentanyl was dissolved with sequential additions of small quantities of heptane solvent with sequential transfers into the mixer. The mixer was stirred until homogeneous. Next, the acrylic, Duro-Tak® 87-901 adhesive and 360 Medical Fluid® additions occurred, followed by the micronized cross-linked polyvinylpyrrolidone, Kollidon® CL-M. Anchor and disperser blade mixing was used throughout the Kollidon® addition. Mixing occurred until a homogeneous mixture was obtained.

The mixture was coated onto ScotchPak® 9744 fluoropolymer release liner at a 9.7 mg/cm² coating weight. The dried coating was laminated to the PET face of the ScotchPak® 9732 backing film, a PET-EVA laminate. The optimum drying condition for coating using a single zone drying oven was 800 RPM Supply and Exhaust fan speed, 5.0 feet per minute line speed, and web temperature equal to 70 to 80° C. Higher web temperatures will melt the dispersed fentanyl API crystals, leading to higher fentanyl skin flux and dissolution shortly after coating. At longer durations, fentanyl crystallizes on the adhesive surface of the coating dried at elevated temperatures leading to poor adhesion, reduced fentanyl drug release and reduced skin flux.

The transdermal patch had the formulation of Table 2.

TABLE 2
Composition % (w/w)

| | Example 1 |
|---|---|
| Component | Composition |
| Fentanyl Base | 4.0% |
| BIO-PSA ® 7-4201 | 85.75% |
| 360 Medical Fluid | 4.5% |
| Kollidon ® CL-M | 4.0% |
| Duro-Tak ® 87-901A | 1.75% |

### Example 2: Comparative Transdermal Patch

A transdermal patch without Kollidon® CL-M was prepared according to the process of Example 1, having the formulation in Table 3. The solvated acrylic polymer separated as large droplets, requiring vigorous agitation to maintain the suspension prior to coating.

TABLE 3
Composition % (w/w)

| | Example 2 |
|---|---|
| Component | Composition |
| Fentanyl Base | 4.0% |
| BIO-PSA ® 7-4201 | 85.75% |
| 360 Medical Fluid | 8.5% |
| Kollidon ® CL-M | 0.0% |
| Duro-Tak ® 87-901A | 1.75% |

### Example 3: Comparative Transdermal Patch

A fentanyl containing transdermal patch was made in accordance with U.S. Pat. No. 8,449,907 to Mylan Pharmaceuticals. The formulation of the transdermal patch of Example 3 presented in Table 4 does not contain Kollidon® CL-M or acrylic adhesive polymer.

TABLE 4
Composition % (w/w)

| | Example 3 |
|---|---|
| Component | Composition |
| Fentanyl Base | 4.0% |
| BIO-PSA ® 7-4201 | 87.50% |
| 360 Medical Fluid | 8.5% |
| Kollidon ® CL-M | 0% |
| Duro-Tak ® 87-901A | 0% |

### Example 4: Comparative Transdermal Patch

A fentanyl containing transdermal patch was made in accordance with Duragesic®. The formulation of the transdermal patch of Example 4 is presented in Table 5. The formulation utilizes an acrylic adhesive polymer, Duro-Tak® 87-4287, in which all fentanyl is dissolved.

TABLE 5
Composition % (w/w)

| | Example 4 |
|---|---|
| Component | Composition |
| Fentanyl Base | 8% |
| Duro-Tak ® 87-4287 | 92% |

### Example 5: Testing

Greater concentrations of the cross-linked, micronized polyvinylpyrrolidone, Kollidon® CL-M, decreased the tack of placebo adhesive compositions while increasing shear (Tables 6a and 6b).

Surprisingly, variation in the Kollidon® to 360 Medical Fluid ratio in the above ranges has little impact on skin flux (Table 7) or peel adhesion (Table 6a). The results of a cold flow study in Table 6b show that increasing amounts of the cross-linked, micronized polyvinylpyrrolidone, Kollidon® CL-M, and decreasing amounts of the 360 Medical Fluid reduce adhesive cold flow. More remarkable still is that the introduction of the acrylic adhesive to the fentanyl, silicone adhesive, Dimethicone oil composition without Kollidon® CL-M (Example 2) does not decrease the skin flux compared to a formulation without the Duro-Tak® 87-901A acrylic adhesive (Example 3), each at the same fentanyl concentration (Table 8). The results in Table 7 show that the variations in the amounts of cross-linked polyvinylpyrrolidone (Kollidon® CL-M) and 360 Medical Fluid also do not affect skin flux.

TABLE 6a
Tack and Shear Results for Varying
360 Medical Fluid; Kollidon Ratios*

| Formulation | 6.0%:2.5% | 4.0%:4.5% | 2.0%:6.5% | 0:8.5% |
|---|---|---|---|---|
| Tack (Newtons, | 3.6 | 3.0 | 2.2 | 1.2 |
| probe tack test)) | | | | |
| Shear(Minutes, 500 | 4 | 16 | 29 | 66 |
| gram weight and 1 | | | | |
| cm²) | | | | |
| Peel Adhesion | 5.7 | 6.0 | 5.3 | 5.9 |
| (Newtons, 1 cm | | | | |
| width) | | | | |

*The BIO-PSA ® 7-4201 is at 85.75% and the Duro-Tak ® 87-901A is at 1.75% in Table 6a formulations. 4% fentanyl is absent in the placebo formulations.

TABLE 6b
Tack and Shear Results for Varying
360 Medical Fluid; Kollidon Ratios*

| Formulation | 7.5%:1.0% | 6.0%:2.5% | 4.5%:4.0% | 3.0%:5.5% |
|---|---|---|---|---|
| Tack (New- | 2.4 | 1.2 | 0.6 | 0.4 |
| tons, probe | | | | |
| tack test)) | | | | |
| Shear(Minutes, | 40 | 66 | 141 | 214 |
| 500 gram | | | | |
| weight and | | | | |
| 1 cm²) | | | | |
| Cold Flow | Significant | Slight | Negligible | Negligible |

*The BIO-PSA ® 7-4201 is at 85.75% and the Duro-Tak ® 87-901A is at 1.75% in Table 6b formulations. 4% fentanyl is absent is all placebo formulations.

TABLE 7
Skin Flux Results - Effect of Varying 360 Medical Fluid: Kollidon ® Ratios

| | Time Points | | | | | | | | |
|---|---|---|---|---|---|---|---|---|---|
| Formulation* | 3 Hrs | 6 Hrs | 9 Hrs | 12 Hrs | 24 Hrs | 36 Hrs | 48 Hrs | 60 Hrs | 72 Hrs |
| Example | 1.34 | 9.19 | 21.54 | 35.5 | 104.4 | 178.2 | 251.3 | 320.0 | 379.8 |
| 1: 4.5% 360 | | | | | | | | | |
| Medical | | | | | | | | | |
| Fluid: | | | | | | | | | |
| 4.0% | | | | | | | | | |
| Kollidon ® | | | | | | | | | |
| CL-M | | | | | | | | | |
| 0% 360 | 0.43 | 6.56 | 18.09 | 32.04 | 103.9 | 187.9 | 271.7 | 348.2 | 419.4 |
| Medical | | | | | | | | | |
| Fluid: | | | | | | | | | |
| 8.5% | | | | | | | | | |
| Kollidon ® | | | | | | | | | |
| CL-M | | | | | | | | | |
| Comparative | 0.89 | 8.28 | 19.75 | 31.13 | 97.00 | 174.5 | 249.0 | 318.4 | 377.4 |
| Example 2 | | | | | | | | | |
| (No | | | | | | | | | |
| Kollidon ®, | | | | | | | | | |
| with 8.5% | | | | | | | | | |
| 360 Medical | | | | | | | | | |
| Fluid) | | | | | | | | | |

*The BIO-PSA ® 7-4201 is at 85.75% and the Duro-Talc ® 87-901A is at 1.75% in all Table 7 formulations.

TABLE 8
Skin Flux Results with and without Acrylic Adhesive
mcg Diffused per area

| | | Example 3 |
|---|---|---|
| Time(hrs) | Example 2 | MYLAN |
| 0 | 0 ± 0 | 0 ± 0 |
| 3 | 0.73 ± 0.52 | 0.23 ± 0.15 |
| 6 | 4.72 ± 2.06 | 2.59 ± 1.67 |
| 9 | 12.46 ± 4.17 | 8.22 ± 4.21 |
| 12 | 22.24 ± 5.31 | 16.04 ± 7.16 |
| 24 | 71.58 ± 12.52 | 61.20 ± 22.94 |
| 36 | 129.71 ± 21.92 | 116.97 ± 39.25 |
| 48 | 189.54 ± 28.52 | 173.27 ± 49.63 |
| 60 | 243.71 ± 37.95 | 233.07 ± 65.64 |
| 72 | 298.32 ± 40.12 | 286.03 ± 76.48 |

### Example 6

Example 6 was prepared according to the process of Example 1. The formulation of the transdermal patch of Example 6 is presented in Table 9.

TABLE 9
Composition % (w/w)

| | Example 6 |
|---|---|
| Component | Composition |
| Fentanyl Base | 4.0% |
| BIO-PSA ® 7-4201 | 85.76% |
| 360 Medical Fluid | 6.5% |
| Kollidon ® CL-M | 2.0% |
| Duro-Tak ® 87-901A | 1.76% |

### Example 7: Comparative

Example 7 was prepared according to the process of Example 1. The formulation of the transdermal patch of Example 7 is presented in Table 10.

TABLE 10
Composition % (w/w)

| | Example 7 |
|---|---|
| Component | Composition |
| Fentanyl Base | 4.0% |
| BIO-PSA ® 7-4201 | 94.1% |
| 360 Medical Fluid | 0% |
| Kollidon ® CL-M | 0% |
| Duro-Tak ® 87-901A | 2.02% |

TABLE 11
Skin Flux Results with and without Kollidon CL-M and 360 Medical Fluid
mcg Diffused per area

| | | | Example 3 |
|---|---|---|---|
| Time(hrs) | Example 6 | Example 7 | MYLAN |
| 0 | 0 ± 0 | 0 ± 0 | 0 ± 0 |
| 3 | 5.76 ± 6.64 | 4.97 ± 5.06 | 4.04 ± 3.50 |
| 6 | 21.40 ± 17.32 | 16.82 ± 12.47 | 18.57 ± 9.04 |
| 9 | 40.37 ± 25.76 | 30.77 ± 17.67 | 35.62 ± 13.93 |
| 12 | 59.69 ± 33.23 | 46.08 ± 22.41 | 53.23 ± 19.06 |
| 24 | 148.42 ± 64.38 | 123.95 ± 43.37 | 137.84 ± 43.31 |
| 36 | 249.79 ± 88.64 | 210.70 ± 60.50 | 236.56 ± 62.43 |
| 48 | 337.00 ± 97.43 | 295.71 ± 65.85 | 330.78 ± 72.67 |
| 60 | 436.56 ± 96.98 | 395.56 ± 68.37 | 432.33 ± 64.83 |
| 72 | 506.06 ± 82.78 | 470.93 ± 62.41 | 511.25 ± 55.84 |

The results in Table 11 further demonstrate that the addition of the cross-linked polyvinylpyrrolidone (Kollidon® CL-F) and the 360 medical fluid do not affect skin flux.

The extent of cold flow of the inventive formulations compared to the Mylan (Example 3) and Duragesic® RLD (Example 4) fentanyl transdermal delivery systems appears summarized in Table 12 below. The improved formulation of this invention demonstrates less cold flow when evaluated under accelerated conditions, placing a 1000 gram weight over 10 cm² circular systems at ambient temperature for one week and measuring the extent of adhesive migration outside the perimeter of the patch.

TABLE 12
Cold Flow Comparison

| System | Cold Flow |
|---|---|
| Comparative Example 4: Duragesic ® RLD | Slight |
| Comparative Example 3: Mylan System | Significant |
| Example 1* | Negligible |

*4% Fentanyl, 85.75% BIO-PSA ® 7-4201, 1.75% Duro-Tak ® 87-901A, 4.5% 360 Medical Fluid, and 4.0% Kollidon ® CL-M.

Procedures:

Skin Flux:

The skin flux measurements occurred at 32°±2° C. using Frans diffusion cells at 600 rpm and pH7.4 PBS with 1.76 in² diffusion surface area.

Peel Adhesion:

A 0.5 inch wide test strip was used for measurement of peel adhesion to stainless steel panels.

Shear Test:

A 500 gram weight was used with a 0.25 in² tape overlap to a stainless steel panel.

Tack:

A 1 mm diameter stainless steel probe was used with the Probe Tack Tester.

See Tables 13 and 14 below for skin flux and dissolution test results immediately following oven drying of Example 1 composition at three different temperatures. The result at a 110° C. temperature setting melts the fentanyl API, yielding an increase in both skin flux and dissolution.

TABLE 13
Drying Temperature Effect on Skin Flux

| | Example 1 | Example 1 | Example 1 | |
|---|---|---|---|---|
| | 80° C. Setting | 110° C. Setting | 83° C. Setting | Example 3 |
| Time(hrs) | (74° C. Web) | (104° C. Web) | (78° C. Web) | MYLAN |
| 0 | 0 | 0 | 0 | 0 |
| 3 | 0.16 | 0.61 | 0.15 | 0.06 |
| 6 | 1.35 | 6.46 | 0.93 | 0.41 |
| 9 | 5.74 | 25.98 | 6.88 | 3.44 |
| 12 | 13.88 | 50.95 | 16.28 | 9.55 |
| 24 | 68.79 | 145.78 | 70.90 | 54.90 |
| 36 | 135.21 | 238.80 | 135.79 | 114.15 |
| 48 | 204.04 | 304.25 | 202.95 | 171.75 |
| 60 | 266.13 | 355.86 | 266.28 | 236.07 |
| 72 | 329.81 | 407.98 | 328.86 | 290.74 |

TABLE 14
Drying Temperature Effect on Dissolution

| | |
|---|---|
| Dissolution Media | 0.05M phosphate buffer pH 2.6 |
| Dissolution Media Volume | 150 mL |
| DPM | 30 |
| Dissolution Apparatus | VII |

| | Example 1 | Example 1 | Example |
|---|---|---|---|
| Time (Hour) | 80° C.-T0 | 110° C.-T0 | 83° C.-T0 |
| 0 | 0 | 0 | 0 |
| 0.5 | 34 | 66 | 36 |
| 1 | 59 | 88 | 61 |
| 2 | 80 | 100 | 83 |
| 4 | 96 | 104 | 98 |
| 12 | 104 | 104 | 106 |
| 24 | 104 | 104 | 107 |

*The preferred drying temperature set point is 80-83° C., corresponding to a web temperature of 74-78° C.

A second aspect of the present invention is directed to a method of relieving pain. In one or more embodiments, the method comprises applying the transdermal delivery system to the skin of a patient in need thereof. As used herein, the term “skin” refers to the outer covering of the body. The skin has multiple layers of ectodermal tissue that guards the underlying muscles, bones, ligaments and internal organs. In one or more embodiments, the transdermal delivery system is applied to the skin, the outer covering of the body, in order to relieve pain. As used herein, the term “patient in need thereof” refers to an individual, a subject, a host, etc. who has a level of pain that needs to be relieved.

The use of the terms “a” and “an” and “the” and similar referents in the context of describing the materials and methods discussed herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A transdermal delivery system comprising:
a backing layer;
an adhesive matrix comprising fentanyl, an analgetically effective relative of fentanyl, or mixtures thereof, a plurality of pressure sensitive adhesives selected from a silicone adhesive, a polyacrylate adhesive, and combinations thereof, and a cross-linked moisture absorbent comprising cross-linked polyvinylpyrrolidone; and
a release liner.

2. The transdermal delivery system of claim 1, wherein the fentanyl comprises crystals having a particle size (d₅₀) in a range of about 5 to 20 microns as measured by laser diffraction.

3. The transdermal delivery system of claim 2, wherein the particle size (d₅₀) is in the range of about 10 to about 15 microns.

4. The transdermal delivery system of claim 1, wherein the polyacrylate adhesive is present in the adhesive matrix in an amount in a range of about 1.0 to about 4.0% w/w.

5. The transdermal delivery system of claim 1, wherein the silicone adhesive is present in the adhesive matrix in an amount in a range of about 80 to about 90% w/w.

6. The transdermal delivery system of claim 1, wherein the fentanyl is present in the adhesive matrix in an amount in a range of about 3 to about 5% w/w.

7. The transdermal delivery system of claim 1, wherein the cross-linked polyvinylpyrrolidone is present in the adhesive matrix in an amount in a range of about 2 to about 6% w/w.

8. The transdermal delivery system of claim 1, wherein the silicone adhesive further comprises a silicone medical fluid.

9. The transdermal delivery system of claim 8, wherein the silicone medical fluid comprises dimethicone.

10. The transdermal delivery system of claim 9, wherein the dimethicone is present in the adhesive matrix in an amount in a range of about 2 to about 7% w/w.

11. The transdermal delivery system of claim 1, wherein the release liner comprises one or more of paper, coated paper, plastic films, polyolefins made of high density polyethylene (HDPE), low density polyethylene (LDPE), polypropylene (PP) plastic resin, fluoropolymer-coated films.

12. The transdermal delivery system of claim 11, wherein the release liner comprises LDPE.

13. The transdermal delivery system of claim 11, wherein the release liner comprises a fluoropolymer-coated polyethylene terephthalate (PET) film.

14. The transdermal delivery system of claim 1, wherein the backing layer comprises films of polyethylene, polyethylene terephthalate (PET), polypropylene, polyurethane, ethylene vinyl acetate (EVA) of polyamide, metal foils, or paper, alone or coated with a polymeric material, or mixtures thereof.

15. The transdermal delivery system of claim 14, wherein the backing layer comprises a PET-EVA laminate.

16. The transdermal delivery system of claim 1, wherein the backing layer has a thickness of less than about 2.5 mil.

17. The transdermal delivery system of claim 1, wherein the transdermal delivery system has reduced cold flow when compared to transdermal delivery systems that do not contain cross-linked polyvinylpyrrolidone.

18. The transdermal delivery system of claim 1, wherein the transdermal delivery system has skin flux in a range of about 2 to about 6 mcg/cm²/h.

19. A transdermal delivery system comprising:
a backing layer;
an adhesive matrix comprising:
3.5 to 4.5% w/w fentanyl,
80 to 90% w/w silicone adhesive,
1 to 4% w/w polyacrylate adhesive, and
2 to 6% w/w cross-linked polyvinylpyrrolidone; and
a release liner.

20. The transdermal delivery system of claim 19, the adhesive matrix further comprising 2 to 7% w/w silicone medical fluid.

21. A method of relieving pain comprising: applying to skin of a patient in need thereof the transdermal delivery system of claim 1.

22. A method of relieving pain comprising: applying to skin of a patient in need thereof the transdermal delivery system of claim 19.
